(19) United States
(12) Patent Application Publication
(10) Pub. No.: US 2012/0051979 A1
(43) Pub. Date: Mar. 1, 2012

(54) CLEANING APPARATUS FOR FOOD PROCESSING EQUIPMENT

(75) Inventors: Sherri D. Clark, St. Charles, MO (US); Michael J. Higgins, St. Charles, MO (US); James L. Marsden, Manhattan, KS (US)

(73) Assignee: HUSSMANN CORPORATION, Bridgeton, MO (US)

(21) Appl. No.: 12/873,025

(22) Filed: Aug. 31, 2010

(51) Int. Cl. A61L 9/20

(52) U.S. Cl. 422/186.3

(57) ABSTRACT

A cleaning apparatus for food processing equipment. The cleaning apparatus includes a base and a curved dome coupled to the base. The base has a first surface with a reflectance, and supports the food processing equipment. The dome is pivotable between an open position and a closed position relative to the base, and includes a second surface having a reflectance. The cleaning apparatus further includes an ultraviolet (“UV”)-based purification device that has at least one photohydroionization (“PHI”) cell operable to produce an advanced oxidation component. The UV-based purification device further has a plurality of UV light sources that are operable to produce UV light. The PHI cell and the UV light sources are coupled to at least one of the base and the dome to impinge directly and indirectly via reflection on the food processing equipment to clean the equipment when the dome is in the closed position.

### BACKGROUND

The present invention relates to a cleaning apparatus, and more particularly, to a cleaning apparatus including one or more lights for cleaning food processing equipment.

Supermarket delis and other retail stores process different meats, cheeses, and other foods using food processing equipment (e.g., deli slicers) throughout the day. Typically, the food processing equipment is not cleaned between slicing operations, much less between uses of the equipment on different food product, which can lead to contamination (e.g., airborne or through physical contact) of the surfaces of the food processing equipment. Often, food processing equipment is cleaned at the end of or at the beginning of the day using liquid anti-microbial wash solutions. Other equipment cleaning methods typically include using fluids with relatively high temperatures, which can make intra-use cleaning of the equipment difficult and time consuming.

### SUMMARY

In one construction, the invention provides a cleaning apparatus for food processing equipment. The cleaning apparatus includes a base and a curved dome coupled to the base and pivotable between an open position and a closed position relative to the base. The base includes a first surface that has a reflectance, and is configured to support the food processing equipment. The dome is defined by an apex and a circumferential edge, and includes a second surface that has a reflectance. The cleaning apparatus also includes an ultraviolet (“UV”)-based purification device that has at least one photohydroionization (“PHI”) cell operable to produce an advanced oxidation component, and a plurality of UV light sources operable to produce UV light. The PHI cell and the UV light sources are coupled to at least one of the base and the dome to impinge directly and indirectly via reflection on the food processing equipment to clean the equipment when the dome is in the closed position.

In another construction, the invention provides a cleaning apparatus for food processing equipment. The cleaning apparatus includes a base and a curved dome coupled to the base and pivotable between an open position and a closed position relative to the base. The base includes a first surface that has a reflectance, and is configured to support the food processing equipment. The dome is defined by an apex and a circumferential edge, and includes a second surface that has a reflectance. The cleaning apparatus also includes a UV-based purification device that has at least one PHI cell coupled to the dome adjacent the apex and operable to produce an advanced oxidation component, and four UV light sources operable to produce UV light. The UV light sources are coupled to the dome and spaced apart from each other. The PHI cell and the UV light sources cooperate to produce an airborne, light-based cleaning agent configured to impinge directly and indirectly via reflection on the food processing equipment to reduce contamination on the food processing equipment when the dome is in the closed position.

In yet another construction, the invention provides a cleaning apparatus for food processing equipment. The cleaning apparatus includes a base and a curved dome coupled to the base and pivotable between an open position and a closed position relative to the base. The base includes a first surface that has a reflectance, and is configured to support the food processing equipment. The dome is defined by an apex and a circumferential edge, and includes a second surface that has a reflectance. The cleaning apparatus also includes a switch and a UV-based purification device that has at least one PHI cell and a plurality of UV light sources. The switch is positioned between the base and the dome and is configured to generate a signal indicative of the dome in the open position and the closed position. The PHI cell is coupled to the dome and is operable to produce an advanced oxidation component. The UV light sources are coupled to at least one of the base and the dome and are operable to produce UV light. The UV-based purification device is configured to activate in response to the dome being in the closed position, and is configured to deactivate in response to the dome being in the open position. The PHI cell and the UV light sources cooperate to produce an airborne, light-based cleaning agent upon activation of the UV-based purification device, and the cleaning agent is configured to impinge directly and indirectly via reflection on the food processing equipment to reduce contamination on the food processing equipment.

Other aspects of the invention will become apparent by consideration of the detailed description and accompanying drawings.

### BRIEF DESCRIPTION OF THE DRAWINGS

FIG. 1 is a perspective view of cleaning apparatus embodying the invention and food processing equipment positioned on a base of the cleaning apparatus.

FIG. 2 is a front view of the cleaning apparatus of FIG. 1 in an open position.

FIG. 3 is a front view with the cleaning apparatus of FIG. 1 in a closed position.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

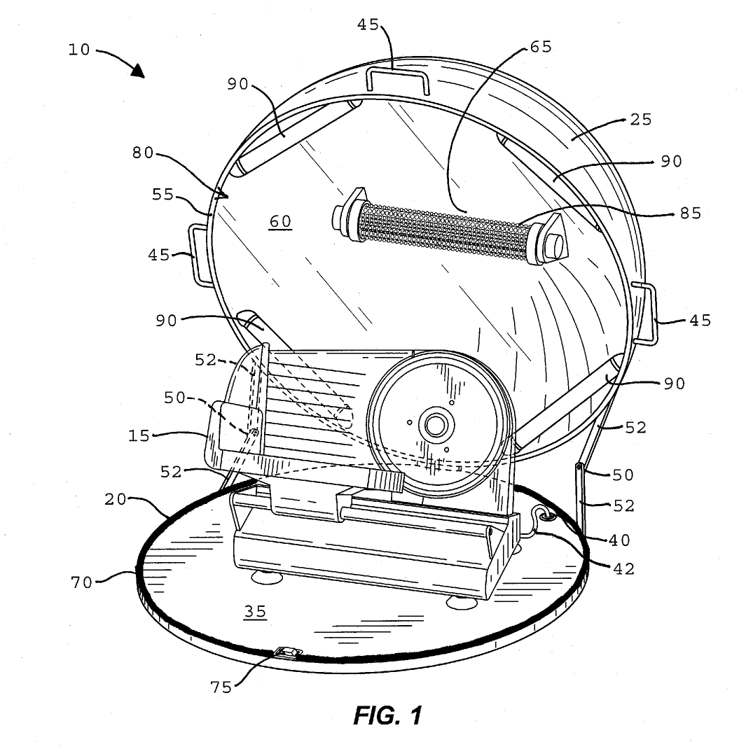

FIGS. 1-3 show a cleaning apparatus 10 for food processing equipment 15 (e.g., food slicers, meat grinders, food processors, blenders, etc.). The cleaning apparatus 10 defines an enclosure or housing for the food processing equipment 15, and the cleaning apparatus 10 can be positioned on any support surface (e.g., floor, countertop, etc.). Generally, the cleaning apparatus 10 is sized to accommodate and substantially enclose or house the food processing equipment 15.

The cleaning apparatus 10 includes a base 20, a lid or dome 25, and a hinge 30. Although the food processing equipment 15 can be removed from the cleaning apparatus 10, the base 20 and the dome 25 generally define a permanent enclosure for the food processing equipment 15. The hinge 30 is coupled between the base 20 and the dome 25 to pivotably attach the dome 25 to the base 20. In other constructions, the dome 25 can be rotatably or pivotably attached to the base 20 by other attachment devices.

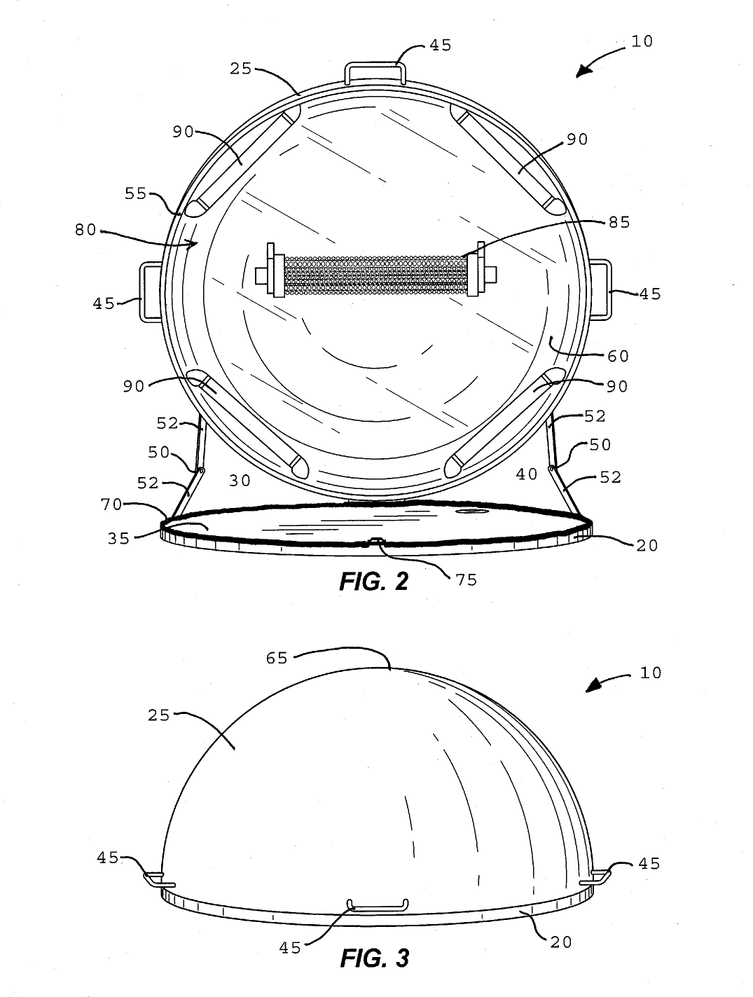

As illustrated in FIGS. 1 and 2, the base 20 includes a substantially flat surface 35 for supporting the food processing equipment 15, and an aperture 40 that extends completely through the base 20 for accommodating passage of a power cord 42 of the food processing equipment 15 through the base 20. In some constructions, the base 20 can include equipment support members (not shown) that are formed on or otherwise coupled to the surface 35 for supporting and/or positioning the food processing equipment 15 within the cleaning apparatus 10. In these constructions, the food processing equipment 15 can be held in place by the equipment support members. In other constructions, the food processing equipment 15 can be secured to the base 20 using fasteners (e.g., bolts, rivets, screws, etc.).

The base 20 can be formed from any material suitable for supporting food processing equipment 15. Generally, the material should be chemically resistant to reduce contamination of the cleaning apparatus 10 and the associated food processing equipment 15. In the illustrated construction, the base 20 is formed from stainless steel such that the surface 35 has a reflectance. In some constructions, the base 20 can include other reflective material (e.g., polished aluminum, mirrors, reflectors, etc.) that forms at least a portion of or is positioned on the surface 35. In other constructions, the base 20 can be formed from an opaque material.

FIGS. 1-3 show that the dome 25 is pivotable between an open position and a closed position relative to the base 20. In general, the open position of the dome 25 corresponds to any position where the dome 25 is not fully closed. Handles 45 are attached to the dome 25 to facilitate movement of the dome 25 between the open position and the closed position. Although the illustrated construction of the cleaning apparatus 10 includes three handles 45, fewer or more than three handles 45 can be attached to the dome 25 to facilitate movement of the dome between the open and closed positions.

Supports 50 are coupled between the base 20 and the dome 25 to support the dome 25 in the open position. In the illustrated construction, each support 50 includes rod portions 52 that pivot relative to each other to move the supports 50 to a hold position when the dome 25 is moved from the closed position to the open position. In some constructions, the supports 50 are lockable in the hold position to inhibit inadvertent or undesired movement of the dome 25 to the closed position. Other support mechanisms for supporting the dome 25 in the open position are possible and considered herein.

The dome 25 is defined by a curved shape, and includes a circumferential or peripheral edge 55, a curved inner surface 60, and an apex 65. In the illustrated construction, the dome 25 has a substantially parabolic shape such that the dome 25 has a focal point or focus adjacent the apex 65. In other constructions, the dome 25 can have a spherical shape, an elliptical shape, or other curved shapes.

In some constructions, the inner surface 60 can be formed from reflective material (e.g., stainless steel, polished aluminum, etc.). In other constructions, a reflector or a mirror or other reflector mechanism can be coupled to or formed as part of the inner surface 60. In other constructions, the dome 25 can include an opaque material.

The cleaning apparatus 10 also includes a gasket 70 that is positioned between the base 20 and the dome 25, and a latch switch 75. The gasket 70 is attached to the base 20 and provides a seal between the base 20 and the dome 25 when the dome 25 is in the closed position such that the cleaning apparatus 10 forms a sealed enclosure around the food processing equipment 15. In other constructions, the gasket 70 can be attached to the dome 25.

In the illustrated construction, the latch switch 75 is coupled to the base 20 and is positioned adjacent a periphery of the base 20. The latch switch 75 generates a signal indicative of the position of the dome 25. In other words, the latch switch 75 indicates whether the dome 25 is in the open position or the closed position. The signal generated by the latch switch 75 can be any signal suitable for indicating the position of the dome 25 (e.g., electronic, mechanical, electro-mechanical, audible, visual, etc.). In other constructions, the latch switch 75 can be coupled to the dome 25.

The cleaning apparatus 10 further includes an ultraviolet (“UV”)-based purification device 80. The UV-based purification device 80 is positioned within the cleaning apparatus 10 to clean the food equipment 15 by directly or indirectly impinging on various surfaces of the food processing equipment 15. As shown in FIGS. 1 and 2, the UV-based purification device 80 includes a photohydroionization (“PHI”) cell 85 and a plurality of UV light sources 90 that cooperate to provide an anti-microbial treatment to the food processing equipment 15. In the illustrated construction, the cleaning apparatus 10 includes one PHI cell 85 and four UV light sources 90. In other constructions, the cleaning apparatus 10 can include any suitable or desired quantity of PHI cells 85 and UV light sources 90 (e.g., more than two PHI cells 85, or fewer or more than four UV light sources 90).

In the illustrated construction, the PHI cell 85 is located at and adjacent the focus of the substantially parabolic-shaped dome 25. In constructions of the cleaning apparatus 10 including a non-parabolic shaped dome 25, the PHI cell 85 can be located at or adjacent the apex 65. Other locations for the PHI cell 85 are also possible and considered herein.

Generally, the PHI cell 85 generates or forms or yields advanced oxidation components (e.g., hydroxyl radicals, ozone, ozone ions, hydroperoxide radicals, hydroxides, hydrogen peroxide, etc.) including oxidizers that react with undesirable compounds on or adjacent the food processing equipment 15 (e.g., contaminants such as microbes, bacteria, odor-causing chemicals, and other inorganic and organic chemicals, etc.), to destroy and/or inactivate the compounds. One such PHI cell 85 can be, for example, the Guardian Air by RGF® produced by RGF Environmental Group, Inc., although other PHI cells 85 may be used in place of the Guardian Air by RGF®.

The UV light sources 90 are spaced apart from each other within the interior of the cleaning apparatus 10 so that UV light emanating from each light source 90 is directed toward the food processing equipment 15. The UV light sources 90 can be located within the cleaning apparatus 10 so that the UV light emanating from the UV light sources 90 directly impinges on the food processing equipment 15, indirectly impinges on the equipment 15, or both. As shown in FIGS. 1 and 2, the UV light sources 90 are located adjacent the circumferential edge 55 of the dome 25 so that UV light can reach the lowermost portion of the food processing equipment 15. In other constructions, the UV light sources 90 can be located elsewhere within the cleaning apparatus 10. For example, one UV light source 90 can be located adjacent the circumferential edge 55, while the remaining UV light sources 90 can be located between the circumferential edge 55 and the apex 65. In other constructions, one or more of the UV light sources 90 can be coupled to the base 20.

In constructions of the cleaning apparatus 10 including reflective surfaces (e.g., the surfaces 35, 60), some portions of UV light from the UV light sources 90 directly impinges on portions of the food processing equipment 15, while other portions of UV light from the UV light sources 90 reflect around the interior of the cleaning apparatus 10 such that the UV light directly and indirectly impinges on portions of the food processing equipment 15. In this manner, a relatively small quantity of UV light sources 90 can be located within the cleaning apparatus 10, thus taking advantage of the reflectance provided by the base 20 and/or the dome 25 without having to position the UV light sources 90 so that the UV light directly impinges on all portions of the food processing equipment 15. The curved shape of the dome 25 further contributes to distributing the UV light toward all areas of the food processing equipment 15. Furthermore, positioning the PHI cell 85 adjacent the apex 65 (e.g., at the focus) of the dome 25 provides a high degree of impingement (directly or indirectly) of the advanced oxidation components on the food processing equipment 15.

When the dome 25 is in the open position, the food processing equipment 15 can be used to process food product (e.g., by slicing, grinding, etc.). During periods where the food processing equipment 15 is inactive or unused, the dome 25 can be pivoted to the closed position. In operation, the latch switch 75 generates the signal indicative of the dome 25 in the closed position. The signal indicative of the dome 25 in the closed position signifies that the food processing equipment 15 housed in the cleaning apparatus 10 can be cleaned. In response to the signal from the latch switch 75, the UV-based purification device 80 is activated. Once the UV-based purification device 80 is activated, the UV light sources 90 generate UV light that reacts with the advanced oxidation components generated by the PHI cell 85 to produce an airborne, light-based cleaning agent. The cleaning agent impinges directly and/or indirectly on the food processing equipment 15 to remove or neutralize contamination on surfaces of the equipment 15.

In the illustrated construction, the cleaning process embodied by the cleaning apparatus 10 is automatic once the dome 25 is in the closed position. In other words, when the dome 25 is closed, the PHI cell 85 and the UV light sources 90 are automatically turned on or otherwise activated in response to the signal from the latch switch 75. In some constructions, the PHI cell 85 and the UV light sources 90 can be selectively activated after the dome 25 is moved to the closed position based one or more factors associated with the cleaning apparatus 10. For example, the UV-based purification device 80 can be activated based on the number of times the dome 25 is opened and closed. Alternatively, the cleaning process can be initiated after a predetermined time period has elapsed and the dome 25 is determined to be in the closed position. In other constructions, the cleaning process can be initiated manually by an operator (e.g., by pushing a button) after the dome 25 is moved to the closed position.

In the illustrated construction, the cleaning process is terminated when the dome 25 is moved from the closed position toward the open position. In other constructions, the cleaning process can be terminated based on other criteria (e.g., manually, after expiration of a predetermined period of time, etc.).

The UV-based purification device 80 utilizes PHI and UV light to provide an anti-microbial cleaning system for the food processing equipment 15. In particular, the cleaning apparatus 10 reduces contamination of the food processing equipment 15 derived from environmental sources and cross-contamination from different foods processed by the same equipment 15, thus reducing food safety risk for foods processed by the equipment 15. The cleaning apparatus 10 fully encloses the food processing equipment 15 so that all of the surfaces of the food processing equipment 15 can be cleaned without complicated, time-consuming cleaning processes embodied by conventional cleaning methods.

The UV-based purification device 80 provides a passive cleaning process for the food processing equipment 15 that can be operated automatically or manually between food processing operations throughout one day of use. The cleaning apparatus 10 inhibits contact contamination from one food to another food during a working day, and uses the air within the cleaning apparatus 10 to reach all of the portions of the food processing equipment 15.

Various features and advantages of the invention are set forth in the following claims.

1. A cleaning apparatus for food processing equipment, the cleaning apparatus comprising:
a base including a first surface having a reflectance, the base configured to support the food processing equipment;
a curved dome coupled to the base and pivotable between an open position and a closed position relative to the base, the dome including a second surface having a reflectance, the dome further including an apex and a circumferential edge; and
an ultraviolet (“UV”)-based purification device including at least one photocatalytic oxidation (“PCO”) cell operable to produce an advanced oxidation component, the UV-based purification device further including a plurality of UV light sources operable to produce UV light, the PCO cell and the UV light sources coupled to at least one of the base and the dome to impinge directly and indirectly via reflection on the food processing equipment to clean the equipment when the dome is in the closed position.

2. The cleaning apparatus of claim 1, wherein at least one of the plurality of UV light sources 90 is coupled adjacent the edge of the dome.

3. The cleaning apparatus of claim 2, wherein four UV light sources spaced apart from each other along the edge of the dome.

4. The cleaning apparatus of claim 1, wherein the curved dome has a substantially parabolic shape.

5. The cleaning apparatus of claim 4, wherein the PCO cell is positioned adjacent the focus of the substantially parabolic-shaped dome.

6. The cleaning apparatus of claim 1, wherein the PCO cell is positioned adjacent the apex of the dome.

7. The cleaning apparatus of claim 1, wherein the enclosure is formed from at least one of stainless steel and polished aluminum.

8. The cleaning apparatus of claim 1, wherein at least one of the plurality of UV light sources is positioned adjacent the circumferential edge of the dome.

9. The cleaning apparatus of claim 1, wherein all of the plurality of UV light sources are positioned adjacent the circumferential edge of the dome.

10. The cleaning apparatus of claim 1, further comprising a switch coupled to one of the base and the dome, wherein the switch is configured to turn on the PCO cell and the UV light sources in response to movement of the dome to the closed position.

11. The cleaning apparatus of claim 1, wherein when the dome is in the closed position, the food processing apparatus is enclosed within the dome.

12. A cleaning apparatus for food processing equipment, the cleaning apparatus comprising:
a base including a first surface having a reflectance, the base configured to support the food processing equipment;
a curved dome defined by an apex and a circumferential edge, the dome coupled to the base and pivotable between an open position and a closed position relative to the base, and the dome including a second surface having a reflectance; and
a UV-based purification device including at least one PCO cell coupled to the dome adjacent the apex and operable to produce an advanced oxidation component, the UV-based purification device further including four UV light sources operable to produce UV light, the UV light sources coupled to the dome and spaced apart from each other, the PCO cell and the UV light sources cooperating to produce an airborne, light-based cleaning agent configured to impinge directly and indirectly via reflection on the food processing equipment to reduce contamination on the food processing equipment when the dome is in the closed position.

13. The cleaning apparatus of claim 12, wherein the curved dome has a substantially parabolic shape.

14. The cleaning apparatus of claim 13, wherein the PCO cell is positioned adjacent the focus of the substantially parabolic-shaped dome.

15. The cleaning apparatus of claim 12, wherein the enclosure is formed from at least one of stainless steel and polished aluminum.

16. The cleaning apparatus of claim 12, wherein all of the plurality of UV light sources are positioned adjacent the circumferential edge of the dome.

17. The cleaning apparatus of claim 12, further comprising a switch coupled to one of the base and the dome, wherein the switch is configured to turn on the PCO cell and the UV light sources in response to movement of the dome to the closed position.

18. A cleaning apparatus for food processing equipment, the cleaning apparatus comprising:
a base including a first surface having a reflectance, the base configured to support the food processing equipment;
a curved dome defined by an apex and a circumferential edge, the dome coupled to the base and pivotable between an open position and a closed position relative to the base, and the dome including a second surface having a reflectance;
a switch positioned between the base and the dome and configured to generate a signal indicative of the dome in the open position and the closed position;
a UV-based purification device including at least one PCO cell coupled to the dome and operable to produce an advanced oxidation component, the UV-based purification device further including a plurality of UV light sources coupled to at least one of the base and the dome and operable to produce UV light, the UV-based purification device configured to activate in response to the dome being in the closed position, and configured to deactivate in response to the dome being in the open position, the PCO cell and the UV light sources cooperating to produce an airborne, light-based cleaning agent upon activation of the UV-based purification device, the cleaning agent configured to impinge directly and indirectly via reflection on the food processing equipment to reduce contamination on the food processing equipment.

19. The cleaning apparatus of claim 18, wherein the PCO cell is coupled to the dome adjacent the apex.

20. The cleaning apparatus of claim 19, wherein all of the UV light sources are attached to the dome adjacent the circumferential edge.
